(19) United States
(12) Patent Application Publication
(10) Pub. No.: US 2025/0058038 A1
(43) Pub. Date: Feb. 20, 2025

(54) CODING MANIFOLD

(71) Applicant: Thomas Vacek, Hampton, VA (US)

(72) Inventor: Thomas Vacek, Hampton, VA (US)

(21) Appl. No.: 18/234,050

(22) Filed: Aug. 15, 2023

(51) Int. Cl. A61M 5/14; A61M 5/168

(52) U.S. Cl. CPC A61M 5/1408; A61M 5/16813; A61M 2005/1403

(57) ABSTRACT

A manifold containing bolus doses of coding or emergency medications. The manifold permits medical personnel to access the medications from a single location, instead of retrieving them separately from various containers. The manifold either stores doses in individual syringes, or stores medications in containers which the doses are retrieved from through the manifold with a single syringe. The manifold is connectable to a venous access line, so the medication can be administered to the patient via the manifold.

### BACKGROUND

In a medical emergency, such as a cardiopulmonary event, the use of medications is exceedingly urgent. Cardiopulmonary events are often referred to in the context of a hospital as “codes” or “coding”. The administration of a “bolus dose”, also known as a single dose administered all at once, through an IV requires retrieval of the emergency medication from the container in which it is stored. When more than one medication is required immediately, each medication must still be retrieved separately from its individual container or canister, and each must be added to the IV line separately. The separate containers may be found on a code cart, in a medication dispensing unit, or even in separate locations from each other, thus requiring additional time to administer all of the necessary medications.

### SUMMARY

A manifold used to provide bolus doses of emergency medications. Each medication has a bolus dose in a container which can be administered using the manifold, permitting the use of a single location to retrieve each required medication. The use of a single location to retrieve the medications permits medical personnel to administer medications more quickly in an emergency, such as a cardiovascular event.

### BRIEF DESCRIPTION OF DRAWINGS

FIG. 1: the manifold, with a single syringe, in a first embodiment

FIG. 2: the manifold, with each container being a syringe, in a second embodiment

### DETAILED DESCRIPTION

In the following description, numerous specific details are set forth to clearly describe the embodiments disclosed herein. However, one skilled in the art will understand that some well known features have not been described in detail so as not to obscure the invention.

In the following description, like reference numbers are used to identify like elements. Furthermore, the drawings are intended to illustrate major features of the exemplary embodiments in a diagrammatic manner. The drawings are not intended to depict every feature of every implementation and are not drawn to scale.

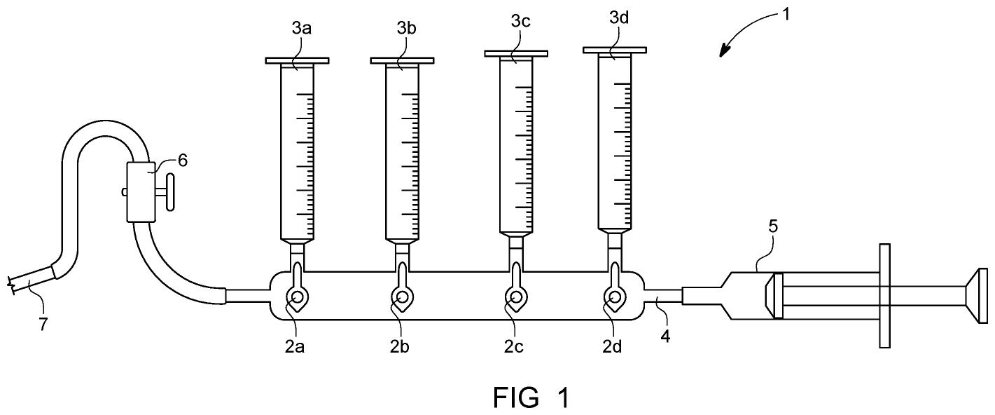

In the first embodiment, as illustrated in figure one, the manifold (1) has a plurality of switches or shutoff valves (2a, 2b, 2c, 2d) and a port (4). Each shutoff provides access to a bolus dose of medication from manifold containers (3a, 3b, 3c, 3d, see FIG. 1). The boluses of the medications may be retrieved through the port using a single syringe (5), with the drug retrieved being determined by the positions of the switches or shutoffs. The opposite end of the manifold from the port has a stopcock (6); this may be connected to a venous access line (7). Specific examples of medications which may be retrieved via the manifold include, but are not limited to, dopamine, noroepinephrine, phenylephrine, saline, and amiodarone.

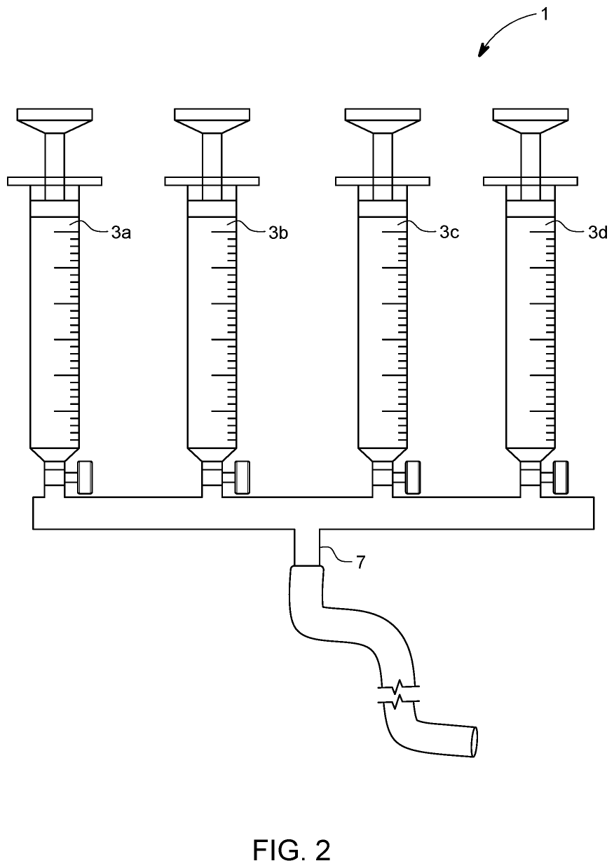

In the second embodiment, as illustrated in FIG. 2, the manifold (1) still has containers (3a, 3b, 3c, 3d), but each container is a syringe. Instead of retrieving the medication through a port and then passing the medication back through the length of the manifold to reach the patient's IV line, the medication is administered directly from the syringe it is stored in with a single pass through the manifold. One end of the manifold is configured to be attached to a venous access line (7). Each syringe has a rubberized plunger, as is known in the art, to make it resistant to accidental discharge of medication.

The manifold may have more or less than the number ports and containers illustrated. The manifold may also be used to offer multiple doses of the same medication, with a single dose being retrieved from a single port. The containers of the first embodiment are standard liquid medication containers, with the thread ordinarily used to hold the lid on being how the containers connect to the manifold. The containers of the second embodiment are also attached via a threaded connection.

The manifold can be placed in crash carts for even quicker access to the medications.

While illustrative embodiments of the invention have been shown and described, variations and alternative embodiments will occur to those skilled in the art. Such variations and alternative embodiments can be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom. Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112(f), unless the element is expressly recited using the exact phrase “means for . . . ” and no method or process step herein is to be construed under the provisions of 35 U.S.C. section 112(f) unless the step, or steps, are expressly recited using the exact phrase “step(s) for . . . ”.

1. A coding manifold configured to permit retrieval of bolus doses of emergency medications, comprising:
connections for at least two containers,
at least two containers, and
a connection to extension tubing configured to connect to an IV line to a patient,
wherein the manifold is configured to permit administration of the bolus doses to the patient through the extension tubing.

2. The coding manifold of claim 1, wherein the containers are each configured to hold one of dopamine, noroepinephrine, phenylephrine, saline, and amiodarone.

3. The coding manifold of claim 1, wherein the connections for at least two containers are threaded.

4. The coding manifold of claim 1, wherein the containers are syringes, and the manifold is configured to permit administration of the bolus doses to the patient through the extension tubing by pressing the plunger on the respective syringe holding the medication being administered.

5. The coding manifold of claim 1, wherein the manifold further comprises:
at least two shutoff valves, and
a port,
wherein each of the connections for at least two containers has a single respective one of the at least two shutoff valves configured to prevent or allow retrieval of the bolus dose of medication from the container through the port via syringe, and the manifold is further configured to transfer the bolus dose of medication from the syringe to the patient through the manifold by appropriate opening and closing of shutoff valves.

6. The coding manifold of claim 1, wherein the manifold is configured to permit air to be flushed out of it using saline from one of the containers.
